(19) United States
(12) Patent Application Publication
(10) Pub. No.: US 2004/0208917 A1
(43) Pub. Date: Oct. 21, 2004

(54) Transdermal systems for the release of clonidine

(76) Inventors: Wilfried Fischer, Vagen (DE); Dirk Schenk, Miesbach (DE)

Correspondence Address: FROMMER LAWRENCE & HAUG, 745 FIFTH AVENUE- 10TH FL., NEW YORK, NY, 10151, US

(21) Appl. No.: 10/417,368

(22) Filed: Apr. 16, 2003

(51) Int. Cl. A61K 9/70

(52) U.S. Cl. 424/449

(57) ABSTRACT

This present invention concerns transdermal systems for the release of clonidine, characterized in that the clonidine is contained in an adhesive layer on the basis of a styrene block polymer, as well as its use in the treatment of hypertonia, migraines, anxieties, hyperkinetic behavioral disorders, alcohol or drug-related withdrawal symptoms, and menopausal symptoms.

This present invention concerns active ingredient-containing transdermal systems (hereinafter referred to as matrix bands or simply as band aids) for the release of clonidine and their use in the treatment of hypertonia, migraines, anxieties, hyperkinetic behavioral disorders, alcohol or drug-related withdrawal systems, and menopausal symptoms.

Active ingredient-containing transdermal systems (hereinafter referred to as “band aids”) have been known to those in the art of pharmaceutical technology for approx. 20 years. Generally speaking, there are two types of technical systems: matrix and reservoir systems. This present invention only concerns matrix systems in which active medical ingredients are directly embedded in a semi-solid matrix made from polymers.

Clonidine is an antisympathetic agent with an imidazoline structure. Clonidine exhibits affinity to 1 and—to a more pronounced extent—to presynaptic and postsynaptic 2 adrenoreceptors and lowers the peripheral sympathic tone. Clonidine primarily causes a drop in blood pressure due to a decreasing heart time-volume and—if administered for longer periods of time—due to a decrease in the peripheral vessel resistance. At the same time, clonidine reduces the release of renine by decreasing angiotensin II in the blood plasma while releasing aldosterone from the cortex of the suprarenal gland.

Clonidine is used for the following indications, among others:

Hypertonia,

Migraines,

Anxieties,

Hyperkinetic behavioral disorders,

Alcohol and drug-related withdrawal symptoms,

Menopausal symptoms.

Clonidine hydrochloride exists as a mesomeric component. Its chemical name is 2-(2,6-dichlorophenyl amino)-2-imidazoline hydrochloride. The molecular formula is C₉H₉Cl₂N₃.HCl. Molecular weight: 266.56.

A number of different clonidine-containing transdermal systems have been developed. U.S. Pat. No. 4,559,222 of Dec. 17, 1995 describes a multilayer transdermal system in which a clonidine base in mineral oil is contained, in a first layer, in a polyisobutylene adhesive together with colloidal silicon dioxide. Onto this layer, a microporous membrane is applied onto which, in turn, an adhesive layer is applied. This adhesive layer is applied to the skin. On the side containing the layer with the active ingredient, the transdermal system is covered by a foil through which clonidine can not pass. This system is disadvantageous considering the poor compatibility of polyisobutylene adhesives with skin, which is known from prior art, its complicated and costly manufacturing process involving many different layers, and the basic physical instability of the system, considering that the layer which comes into contact with the skin is saturated with clonidine over time, as a result of which the release properties of the system change, i.e. a system which has been stored for a longer period of time releases the active ingredient from the contact layer faster than this ingredient can be resupplied by the microporous membrane. Another disadvantage of such a system is the poor adhesive force of the system. Considering that the transdermal system must be carried by patients for seven days, the manufacturer must also provide an active ingredient-free band to be applied onto the system containing clonidine to ensure that it will stay in place properly and securely, which further increases costs and causes complications for the user.

U.S. Pat. No. 5,762,952 of Jun. 9, 1988 describes an improved system comprising a self-crosslinking acrylate adhesive wherein, for example, clonidine has been added together with auxiliary agents which are volatile at higher temperatures, such as solvents or resorption promoters. Crosslinking is necessary to increase the consistency of the adhesive mass, which is reduced to such an extent by adding large quantities of liquid components such as solvents or resorption promoters that no coherent adhesive layer is obtained any longer. Disadvantages of this invention include the use of toxic crosslinking agents as well as potentially skin-irritating solvents and resorption promoters.

U.S. Pat. No. 5,958,446 describes an invention wherein a mixture of self-adhesive acrylates and polyisobutylene or silicones produce a flow through the skin which is higher than is achieved when polymers alone are used. Although above patent claims the use of clonidine as an active agent, no example for such use is described. This invention is disadvantageous insofar as the combination of two polymers in the majority of examples that are described (e.g. with 17β estradiol, norethisterone acetate, pilocarpine, all substances which penetrate the skin well) is produced by using resorption promoters such as lecithin or propylene glycol to achieve a sufficient flow rate. This means that the use of the mixtures of polymers described in this patent alone is not sufficient to produce transdermal systems with sufficient efficiency.

The object of this present invention is therefore to provide a transdermal system for the release of clonidine which can be produced in a very inexpensive manner, is very well tolerated by the skin, is easy to use for patients, does not require any additional means of fixation, releases between 100 and 300 μg of clonidine per day through the skin, and does not contain any toxic crosslinking agents or solvents/resorption promoters.

In accordance with this present invention, this is achieved by a transdermal system for the release of clonidine as described below:

In accordance with one embodiment hereof, this present invention concerns a transdermal system for the release of clonidine comprising a clonidine-containing adhesive layer on the basis of a styrene block polymer and/or styrene block copolymer.

In the transdermal system in accordance with this present invention, the styrene block polymer may be a styrene butadiene block copolymer or a styrene butadiene styrene block copolymer or one of their mixtures.

In addition, in the transdermal system in accordance with this present invention, the styrene block polymer and/or the styrene block copolymer are not crosslinked.

Furthermore, in the transdermal system in accordance with this present invention, the styrene block copolymer may comprise a non-saturated elastomer block, which is preferably not in the end position.

In addition, in the transdermal system in accordance with this present invention, the adhesive layer may contain clonidine in a concentration ranging from 0.1 to 20 percent by weight.

Preferably, the adhesive layer may contain clonidine in a concentration ranging from 2 to 10 percent by weight.

In addition, in the transdermal system in accordance with this present invention, the adhesive layer may contain, in addition to clonidine and the styrene block polymer and/or styrene block copolymer, the following:

Filling agents, and/or

Skin-protecting agents, and/or

Softeners, and/or

Tackifiers.

As a softener, paraffin may be used, in particular viscous paraffin or paraffin oil.

In addition, the softener may be present in a quantity of up to 15 percent by weight, in particular up to 10 percent by weight, and preferably up to 7 percent by weight, based on matrix weight.

As a tackifier or as an additional tackifier, a colophonium derivative may be used, preferably foral.

In addition, the tackifier or the additional tackifier may be present in a quantity of up to 8 percent by weight, in particular in a quantity of up to 6 percent by weight, based on matrix weight.

In addition, in the transdermal system in accordance with this present invention, the adhesive layer containing clonidine may form one layer of a flat, self-adhesive band with a multilayer structure.

In addition to the adhesive layer containing clonidine, the transdermal system may comprise a cover layer and, on the side facing away from the cover layer, a carrier layer which is removable and which temporarily covers the adhesive layer.

In accordance with another embodiment hereof, this present invention concerns a transdermal system for the release of clonidine comprising a clonidine-containing adhesive layer, wherein the adhesive layer comprises two layers (sublayers) or is provided in two layers, and each layer is based on a styrene block polymer and/or styrene block copolymer.

In the transdermal system in accordance with this present invention, the styrene block polymer may be a styrene butadiene block copolymer or a styrene butadiene styrene block copolymer or one of their mixtures.

In addition, in the transdermal system in accordance with this present invention, the styrene block polymer and/or the styrene block copolymer are not crosslinked.

Furthermore, in the transdermal system in accordance with this present invention, the styrene block copolymer may comprise a non-saturated elastomer block, which is preferably not in the end position.

In addition, in the transdermal system in accordance with this present invention, the adhesive layer may contain clonidine in a concentration ranging from 0.1 to 20 percent by weight.

The adhesive layer may contain clonidine in a concentration ranging from 2 to 10 percent by weight.

In addition, in the transdermal system in accordance with this present invention, the sublayer of the adhesive layer facing the skin may contain a different, e.g. a higher, concentration of clonidine than the adjacent layer(s) of the adhesive layer which face away from the skin.

In addition, in the transdermal system in accordance with this present invention, the adhesive layer may contain, in addition to clonidine and the styrene block polymer and/or styrene block copolymer, the following in the sublayer facing the skin and in the layer(s) facing away from the skin of the adhesive layer:

Filling agents, and/or

Skin-protecting agents, and/or

Softeners, and/or

Tackifiers.

As a tackifier, a colophonium derivative may be used, preferably foral.

In addition, in the transdermal system in accordance with this present invention, in the sublayer of the adhesive layer which faces the skin, as an additional tackifier, a colophonium derivative may be used, preferably foral.

The tackifier and/or the additional tackifier may each be present in a quantity of up to 8 percent by weight, in particular in a quantity of up to 6 percent by weight, based on matrix weight.

In addition to the adhesive layer containing clonidine, the transdermal system may comprise a cover layer and, on the side facing away from the cover layer, a carrier layer which is removable and which temporarily covers the adhesive layer.

In addition, in the transdermal system in accordance with this present invention, the dry adhesive layer may have an areal weight of between 20 and 150 g/m².

In addition, in the transdermal system in accordance with this present invention, the dry adhesive layer may have an areal weight of between 50 and 120 g/m².

In addition, in the transdermal system in accordance with this present invention, the cover layer may be made from plastic foil, plastic foam, woven fabric, or fleece.

In addition, in the transdermal system in accordance with this present invention, the carrier layer may be made from plastic foil, paper, or a laminate thereof.

In addition, in the transdermal system in accordance with this present invention, the carrier layer may, at least on the side facing the adhesive layer, be provided with a separating means, which is preferably siliconized (siliconization) or metallized (metallization).

The plastic foil may be a polyester, polyethylene, or polypropylene foil.

In addition, in the transdermal system in accordance with this present invention, the release rate may be between 10 and 1000 μg of clonidine per day.

For example, the release rate may be between 50 and 500 μg of clonidine per day.

Finally, in accordance with one embodiment hereof, this present invention concerns the use of a transdermal system in accordance with this present invention in the treatment of hypertonia, migraines, anxieties, hyperkinetic behavioral disorders, alcohol or drug-related withdrawal symptoms, and menopausal symptoms.

This present invention is based on the surprising findings that a pressure-sensitive adhesive glue on the basis of a styrene block polymer and/or styrene block copolymer, for example a styrene butadiene styrene block copolymer, meets all of the above requirements: a sufficient concentration of the clonidine base is soluble in the dried adhesive, and the chemical potential of clonidine in the dried adhesive is high enough without adding further components to maintain a sufficient flow of the active ingredient through intact skin for a period of seven days. The adhesive does not require any further crosslinking agents to achieve optimum consistency together with the clonidine which is dissolved therein. The adhesive properties are such that an excellent adhesive force is obtained for seven days without producing significant skin irritations. The use of additional means to secure the band is no longer required.

The following is a detailed description of this present invention, which is not intended to limit its scope in any manner whatsoever.

Clonidine bands are produced by using conventional machinery that is known to those in the art.

The clonidine base is dissolved or dispersed in a suitable, highly volatile solvent, such as ethyl acetate, ethanol, isopropanol, dioxane, or ethyl methyl keton. The solution/dispersion is mixed with a solution of the above-described pressure-sensitive adhesive in a suitable vessel. Optionally, although without being necessarily required, common substances such as filling agents, skin-protecting agents, and/or tackifiers may be added. The mixture of clonidine and styrene block polymers and/or styrene block copolymers, e.g. styrene butadiene styrene block copolymers and, optionally, further substances is applied by using a conventional coating machine to a substrate and/or a carrier, e.g. siliconized plastic foils or siliconized paper or the like, and the solvent is removed in a drier located downstream therefrom. After leaving the drier, the dried and self-adhesive active ingredient/adhesive matrix is lined with another layer, which may, for example, be a plastic foil, a fleece, a plastic foam, or a woven fabric, which is used to cover the adhesive matrix.

In another processing step, by using a cutting or punching device known to those in the art, the desired transdermal systems can be cut or punched out in specified shapes and sizes. For protection purposes, the finished systems can be placed in bags or other similar packages.

In order to characterize transdermal systems in terms of the release of the active ingredient, two main methods are used:

1. In-vitro skin permeation tests.

2. In-vitro release tests in accordance with pharmacopoeias currently in effect.

Skin permeation tests are frequently conducted by using the isolated skin of naked mice. A piece of a band is applied to the top side of the skin and mounted in a diffusion cell. A buffer solution (acceptor) comes into contact with the bottom side of the skin, and the time-dependent change in concentration in the acceptor medium is measured. The results obtained for the preparations in accordance with this present inventions are presented in the examples below.

In-vitro release tests are performed in glass vessels built in accordance with the provisions of the pharmacopoeias. In a cylindrical 1 l vessel with a round bottom, the band is secured on a perforated plate in such a manner that the adhesive layer faces upwards. The perforated plate is placed on the bottom of the vessel, and the vessel is filled with water. Afterwards, a specific stirrer is used to achieve a uniform concentration. Again, the time-dependent concentration in the medium in which the release occurs is measured. The results of these tests are shown in the examples below.

These methods differ insofar as the release tests only consider the release of the active ingredient from the band, which usually is not correlate with its biological effect. The skin permeation model, however, also considers, in addition to the required release, the distribution of the active ingredient in the skin as well as its diffusion through the skin. As a general rule, this can be correlated with the biological effect of the active ingredient.

The following examples are intended to illustrate this present invention, without limiting its scope in any manner whatsoever.

### COMPARATIVE EXAMPLE 1

A commercially available clonidine band, Catapres® TTS, with the following characteristics:

| | | |
|---|---|---|
| Clonidine content: | 5 | mg |
| Area: | 7 | cm² |

Composition (qualitative):

Mineral oil

Polyisobutylene

Colloidal silicon dioxide

Microporous polypropylene membrane

was subjected to in-vitro dissolution testing as per the European Pharmacopoeia. The results are shown in Table 1.

In addition thereto, in-vitro skin permeation was tested in a mouse skin model.

Procedure:

A 1.5 cm² piece of skin of female naked mice, from which the subcutaneous tissue had been removed, is placed on the opening, measuring precisely 1 cm², of an automated diffusion cell, a piece of the clonidine band measuring approx. 1.5 cm² is applied thereto and sealed off on the cell by using a contact pressure device. Afterwards, the cell is filled with 25 ml of a physiological HEPES buffer solution, and the temperature is set to 34° C. At predefined times, samples are taken from the buffer solution, and the concentration of active ingredient contained therein is determined by high-pressure liquid chromatography.

All bands described below are tested by using this testing procedure.

The results obtained are shown in Table 2.

TABLE 1
In-Vitro Release of a Clonidine Transdermal System
(Matrix Band)

| Time | Comparative Example 1 |
|---|---|
| (Hours) | (Release of Clonidine, in Percent) |
| 2 | 10.44 |
| 4 | 11.82 |
| 24 | 20.95 |

TABLE 2
In-Vitro Skin Permeation of a Clonidine Transdermal System
(Matrix Band)

| Time | Comparative Example 1 (Permeation |
|---|---|
| (Hours) | of Clonidine, in μg/cm²) |
| 3 | 24.0 |
| 6 | 56.0 |
| 9 | 80.5 |
| 14 | 113.5 |
| 19 | 139.0 |
| 24 | 163.5 |
| 32 | |
| 36 | 233.5 |
| 40 | |
| 48 | 305.5 |

### COMPARATIVE EXAMPLE 2

In this Comparative Example, the matrix had the following composition:

| | |
|---|---|
| Clonidine base | 5.0% |
| Foral | 8.5% |
| Duro-Tak 387-4098 | 86.5% |

Clonidine base (Leiras, Finland) was dissolved in ethyl acetate (Merck, Germany), and foral (E 105, Hercules, Netherlands) was added. The solution was mixed with the Duro-Tak 387-4098 solution, and a thin foil was applied to the siliconized side of a polyester release liner (FL 2000, Loparex, Netherlands). After drying the ethyl acetate in a conventional coating machine, an active ingredient-containing adhesive layer with an areal weight of 100 g/m² was obtained. The matrix was laminated with a polyester foil (Hostaphan, Mitsubishi Polyester Foils, Germany), and 10 cm² were punched out, which were packaged in aluminum bags and stored at temperatures of 4° C. and 40° C. The content of the clonidine release product and/or the lonidine were measured by means of high-pressure liquid chromatography (HPLC).

| Test | Storage | Start | 1 Week | 1 Month |
|---|---|---|---|---|
| Clonidine | 4° C. | 0.07 | 0.22 | ./. |
| Release | 40° C. | 0.07 | 0.97 | 1.5 |

Even at a temperature of 4° C., within one week, a significant increase in clonidine release can be observed, and at a temperature of 40° C., this rate already reaches an inadmissibly high value after a period of one month.

### EXAMPLE 1

A stable, double-layer band has the following composition

| | |
|---|---|
| Clonidine base | 6.0% |
| Paraffin-viscous | 10.0% |
| Duro-Tak 87-6173 | 84.0% |

By using the same procedure describe above for Comparative Example 2, a laminate is produced whose active ingredient-containing adhesive matrix has an areal weight of 60 g/m². As a release liner with a siliconized side, a polyester foil (FL 2000, Loparex, Netherlands) is used. Such laminate is rolled up and used for the subsequent production step instead of the backing foil (Hostaphan).

2^{nd} Layer

| | |
|---|---|
| Clonidine base | 3.0% |
| Foral-105 E | 5.0% |
| Paraffin-viscous | 10.0% |
| Duro-Tak 87-6173 | 82.0% |

This solution is also applied to the release liner (FL 2000), at a dry weight of 60 g/m², and laminated together on the coating machine with the first layer in such a manner that the two adhesive matrixes are glued together. For that purpose, the release line of the first layer is removed by the machine prior to lamination. Afterwards, it is laminated together with the backing foil. A laminate comprising a backing foil and a double-layer active ingredient-containing adhesive matrix is obtained whose layer facing the backing foil does not contain any foral and whose layer facing the skin, however, contains a lower concentration of clonidine. The purpose thereof is to provide a layer facing the skin which controls the release from the matrix through foral content, whereas the layer facing the backing foil acts as a reservoir for long-term application beyond the first week as a result of the higher clonidine content.

In this case, the duro-tak glue is a styrene butadiene styrene copolymer (SBS). Foral is used as a tackifier, while paraffin is used as a softener.

The stability of the formulation in terms of acetyl clonidine is shown in the table below:

| | | Start | 3 Months | 15 Months |
|---|---|---|---|---|
| Clonidine | 5 ° C. | n. d. | — | — |
| Release | | | | |
| | 25° C./60° C. | | 0.01% | 0.01% |
| | 30° C./60° C. | | 0.01% | 0.02% |
| | 40° C./75° C. | | 0.02% | 0.03% |

### EXAMPLE 2

A non-crosslinked glue of the type styrene block copolymer with an elastomeric middle block, unsaturated, comprising polybutadiene (SBS, styrene butadiene styrene) was used. Such a band has the following composition:

| | | |
|---|---|---|
| Content: | Clonidine base | 6.0% |
| | Foral | 5.0% |
| | Paraffin oil | 5.0% |
| | Duro-Tak 87-6174 | 84.0% |
| | Spatial weight: | 140 g/m², 10 cm² |

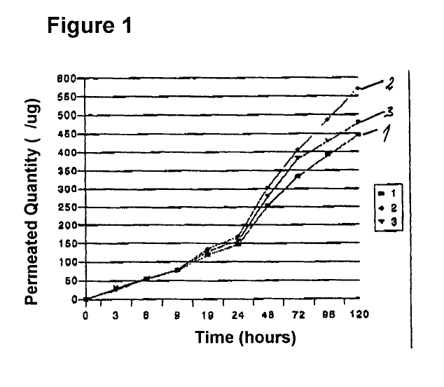

The main permeation data are graphically shown in FIG. 1.

Permeated Quantity (g of Clonidine)

| Time | | | |
|---|---|---|---|
| (Hours) | 1 | 2 | 3 |
| 0 | 0 | 0 | 0 |
| 3 | 30.75 | 27.98 | 25.40 |
| 6 | 55.84 | 56.28 | 54.47 |
| 9 | 76.92 | 80.80 | 78.15 |
| 19 | 119.60 | 134.92 | 128.53 |
| 24 | 146.87 | 168.51 | 156.60 |
| 48 | 252.52 | 302.80 | 277.41 |
| 72 | 332.92 | 405.80 | 381.76 |
| 96 | 391.11 | 486.53 | 428.00 |
| 120 | 445.27 | 570.02 | 477.17 |

1. A transdermal system for the release of clonidine with a clonidine-containing adhesive layer on the basis of polymers from the group of styrene block polymers, styrene block copolymers, and their combinations.

2. The transdermal system in accordance with claim 1, characterized in that the styrene block copolymer is a styrene butadiene block copolymer or a styrene butadiene styrene block copolymer or one of their mixtures.

3. The transdermal system in accordance with claims 1 and/or 2, characterized in that the styrene block polymer and/or the styrene block copolymer are not crosslinked.

4. The transdermal system in accordance with at least one of the preceding claims, characterized in that the styrene block copolymer comprises a non-saturated elastomer block, which is preferably not in the end position.

5. The transdermal system in accordance with at least one of the preceding claims, characterized in that the adhesive layer contains clonidine in a concentration ranging from 0.1 to 20 percent by weight.

6. The transdermal system in accordance with claim 5, characterized in that the adhesive layer contains clonidine in a concentration ranging from 2 to 10 percent by weight.

7. The transdermal system in accordance with at least one of the preceding claims, characterized in that the adhesive layer contains, in addition to clonidine and the polymers of the adhesive layer, at least one of the following:
Filling agents,
Skin-protecting agents,
Softeners,
Tackifiers, and
their combinations.

8. The transdermal system in accordance with claim 7, characterized in that, as a softener, paraffin is used, in particular viscous paraffin or paraffin oil.

9. The transdermal system in accordance with claim 8, characterized in that the softener is present in a quantity of up to 15 percent by weight, in particular up to 10 percent by weight, and preferably up to 7 percent by weight, based on matrix weight.

10. The transdermal system in accordance with at least one of claims 7 through 9, characterized in that as a tackifier or as an additional tackifier, a colophonium derivative is used, preferably foral.

11. The transdermal system in accordance with claim 10, characterized in that the tackifier, the additional tackifier, or the tackifier and the additional tackifier are present in a quantity of up to 8 percent by weight each, in particular in a quantity of up to 6 percent by weight each, based on matrix weight.

12. The transdermal system in accordance with at least one of the preceding claims, characterized in that the adhesive layer containing clonidine forms one layer of a flat, self-adhesive band with a multilayer structure.

13. The transdermal system in accordance with claim 12, characterized in that the transdermal system comprises, in addition to the clonidine-containing adhesive layer, a cover layer and, on the side facing away from the cover layer, a carrier layer which is removable and which temporarily covers the adhesive layer.

14. The transdermal system in accordance with at least one of the preceding claims, characterized in that the dry adhesive layer has an areal weight of between 20 and 150 g/m².

15. The transdermal system in accordance with claim 14, characterized in that the dry adhesive layer has an areal weight of between 50 and 120 g/m².

16. The transdermal system in accordance with at least one of claims 13 through 15, characterized in that the cover layer is made from plastic foil, plastic foam, woven fabric, or fleece.

17. The transdermal system in accordance with at least one of claims 13 through 16, characterized in that the carrier layer is made from plastic foil, paper, or a laminate thereof.

18. The transdermal system in accordance with at least one of claims 13 through 17, characterized in that the carrier layer is, at least on the side facing the adhesive layer, be provided with a separating means, which is preferably siliconized (siliconization) or metallized (metallization).

19. The transdermal system in accordance with at least one of claims 13 through 18, characterized in that plastic foil is a polyester, polyethylene, or polypropylene foil.

20. The transdermal system in accordance with at least one of the preceding claims, characterized in that the release rate is between 10 and 1000 μg of clonidine per day.

21. The transdermal system in accordance with claim 20, characterized in that the release rate is between 50 and 500 μg of clonidine per day.

22. A use of a transdermal system in accordance with at least one of the preceding claims in the treatment of hypertonia, migraines, anxieties, hyperkinetic behavioral disorders, alcohol or drug-related withdrawal symptoms, and menopausal symptoms.

23. The transdermal system for the release of clonidine with a clonidine-containing adhesive layer, wherein the adhesive layer is provided in the form of two layers (sublayers) or comprises two layers, characterized that each sublayer is manufactured on the basis of polymers from the group of styrene block polymers, styrene block copolymers, and their combinations.

24. The transdermal system in accordance with claim 23, characterized in that the styrene block copolymer is a styrene butadiene block copolymer or a styrene butadiene styrene block copolymer or one of their mixtures.

25. The transdermal system in accordance with claims 23 and/or 24, characterized in that the styrene block polymer, the styrene block copolymer, or the styrene block polymer and the styrene block copolymer are not crosslinked.

26. The transdermal system in accordance with at least one of claims 14 through 25, characterized in that the styrene block copolymer comprises a non-saturated elastomer block, which is preferably not in the end position.

27. The transdermal system in accordance with at least one of claims 14 through 26, characterized in that the adhesive layer contains clonidine in a concentration ranging from 0.1 to 20 percent by weight.

28. The transdermal system in accordance with claim 27, characterized in that the adhesive layer contains clonidine in a concentration ranging from 2 to 10 percent by weight.

29. The transdermal system in accordance with at least one of claims 23 through 28, characterized in that the sublayer of the adhesive layer facing the skin may contain a different, e.g. a higher, concentration of clonidine than the adjacent layer(s) of the adhesive layer facing away from the skin.

30. The transdermal system in accordance with at least one of claims 23 through 29, characterized in that the adhesive layer contains, in addition to clonidine and the polymers of the adhesive layer, at least one of the following in the sublayer facing the skin and in the layer(s) facing away from the skin of the adhesive layer:
Filling agents,
Skin-protecting agents,
Softeners,
Tackifiers, and
their combinations.

31. The transdermal system in accordance with at least one of claims 23 through 30, characterized in that, as a tackifier, a colophonium derivative is used, preferably foral.

32. The transdermal system in accordance with claim 30, characterized in that, in the sublayer of the adhesive layer which faces the skin, as an additional tackifier, a colophonium derivative is used, preferably foral.

33. The transdermal system in accordance with claims 31 and/or 32, characterized in that the tackifier, the additional tackifier, or the tackifier and the additional tackifier may each be present in a quantity of up to 8 percent by weight each, in particular in a quantity of up to 6 percent by weight each, based on matrix weight.

34. The transdermal system in accordance with at least one of claims 23 through 33, characterized in that the transdermal system comprises, in addition to the clonidine-containing adhesive layer, a cover layer and, on the side facing away from the cover layer, a carrier layer which is removable and which temporarily covers the adhesive layer.

35. The transdermal system in accordance with at least one of the preceding claims, characterized in that the dry adhesive layer has an areal weight of between 20 and 150 g/m².

36. The transdermal system in accordance with claim 35, characterized in that the dry adhesive layer has an areal weight of between 50 and 120 g/m².

37. The transdermal system in accordance with at least one of claims 23 through 36, characterized in that the cover layer is made from plastic foil, plastic foam, woven fabric, or fleece

38. The transdermal system in accordance with at least one of claims 23 through 37, characterized in that the carrier layer is made from plastic foil, paper, or a laminate thereof.

39. The transdermal system in accordance with at least one of claims 23 through 38, characterized in that the carrier layer is, at least on the side facing the adhesive layer, provided with a separating means, which is preferably siliconized (siliconization) or metallized (metallization).

40. The transdermal system in accordance with at least one of claims 23 through 39, characterized in that the plastic foil is a polyester, polyethylene, or polypropylene foil.

41. The transdermal system in accordance with at least one of claims 23 through 40, characterized in that the release rate is between 10 and 1000 μg of clonidine per day.

42. The transdermal system in accordance with claim 41, characterized in that the release rate is between 50 and 500 μg of clonidine per day.

43. The use of a transdermal system in accordance with at least one of claims 23 through 42 for the treatment of hypertonia, migraines, anxieties, hyperkinetic behavioral disorders, alcohol or drug-related withdrawal symptoms, and menopausal symptoms.
